(19) United States
(12) Patent Application Publication
(10) Pub. No.: US 2003/0097103 A1
(43) Pub. Date: May 22, 2003

(54) Absorbent article

(76) Inventors: James Cameron Horney, Cincinnati, OH (US); John Richard Noel, Cincinnati, OH (US); Sheri Dean Keeler, West Harrison, IN (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/990,663

(22) Filed: Nov. 21, 2001

(51) Int. Cl. A61F 13/00; A61F 15/00; A61F 13/15; A61F 13/20

(52) U.S. Cl. 604/369; 604/365

(57) ABSTRACT

An absorbent article comprises an open-cell foam, which provides the absorbent structure thereof. The absorbent structure has a means for enhancing transport of the fluids from the upper surface into the core region, the means being selected from: a) localized expanded regions of the foam, b) apertures in the foam; c) integration to a topsheet and d) combinations of (a), (b), and (c). Also disclosed are processes of treating thin-after-drying foam absorbent materials to provide selective expansion, aperturing, and topsheet integration.

### BACKGROUND ART

The following references relate to absorbent structures: U.S. Pat. No. 2,747,575, issued May 29, 1956, in the name of Mercer; U.S. Pat. No. 4,425,130, issued Jan. 10, 1984, in the name of DesMarais; U.S. Pat. No. 4,950,264, issued Aug. 21, 1990, in the name of Osborn, III; U.S. Pat. No. 5,009,653, issued Apr. 23, 1991, in the name of Osborn, III; PCT Pat. Publication WO 94/16658, published Aug. 4, 1994, in the names of Osborn, III, et al.; U.S. Pat. No. 4,804,380, issued Feb. 14, 1989, in the names of Lassen et al.; European Pat. App. EP 0 804 915 A1, published Nov. 5, 1997, in the names of Carlucci et al.; European Pat. App. EP 0 804 917 A1, published Nov. 5, 1997, in the names of Carlucci et al.; U.S. Pat. No. 4,110,276, issued Aug. 29, 1978, in the name of DesMarais; U.S. Pat. No. 4,752,349, issued Jun. 21, 1988, in the name of Gebel; U.S. Pat. No. 4,409,592, issued Oct. 11, 1983, in the name of Hunt; U.S. Pat. No. 5,849,805, issued Dec. 15, 1998, in the name of Dyer; U.S. Pat. No. 5,899,893, issued May 4, 1999, in the names of Dyer et al.; and U.S. Pat. No. 5,873,869, issued Feb. 23, 1999, in the names of Hammons et al.

### FIELD OF THE INVENTION

The present invention relates to absorbent articles comprising open cell foams. The absorbent structures provided by the open cell foams have means for enhancing transport of body fluids from the upper surface to the core region, the means being selected from localized expanded regions of the foam, apertures in the foam and combinations of these features.

### BACKGROUND OF THE INVENTION

Absorbent articles, such as sanitary napkins, panty liners, and incontinence pads, are devices that are typically worn in the crotch region of an undergarment. These devices are designed to absorb and retain liquid and other discharges from the human body and to prevent soiling of the wearer's body and clothing. Sanitary napkins and panty liners are a type of absorbent article usually worn by women. A wide variety of shapes and dimensions of such articles are currently used by women for the collection of menses and other bodily discharges.

In the past a number of efforts have been directed to providing absorbent articles that maintain contact with the wearer's body. For example, one attempt to provide such body contact employs a catamenial bandage having a longitudinal hump, which bulges towards and may contact the body of the wearer.

It is also desirable that sanitary napkins, not only maintain contact with, but also conform as closely as possible to the wearer's body. Such a body-conforming capability increases the effectiveness of the sanitary napkin, by reducing the possibility that menses will travel beyond the perimeter of the sanitary napkin and leak. A generally thin, flexible sanitary napkin, with or without a central absorbent hump, is conformable and is capable of handling medium to high menstrual flows.

Other sanitary napkins comprise an expanding layer, comprised of regenerated cellulose sponge, which, when activated by body fluids, expands into a tridimensional structure. The expanding layer has apertures into its body facing surface and/or its garment-facing surface.

The development of highly absorbent articles for blood and blood-based liquids, such as catamenial pads (e.g., sanitary napkins) tampons, wound dressings, bandages and surgical drapes can be challenging. Compared to water and urine, blood and blood-based liquids, such as menses, are relatively complex mixtures of dissolved and undissolved components (e.g., red blood cells). In particular, blood based liquids, such as menses, are much more viscous than water and urine. This higher viscosity hampers the ability of conventional absorbent materials to efficiently and rapidly transport these blood-based liquids to regions remote from the point of initial discharge. Undissolved elements in these blood-based liquids can also potentially clog the capillaries of these absorbent materials. This makes the design of appropriate absorbent systems for blood-based liquids, such as menses, particularly difficult.

Foams of various types have been suggested for use in tampons, sanitary napkins, and other articles that absorb blood and blood-based liquids. Among these foams are soft, flexible open celled foams made from polyurethanes, cellulose or styrenetbutadiene rubber, foams of “medium cell size” hydrophilized by surfactant treatment and having a density within the range of 0.1 to 0.8 g/cc, and biodegradable hydrophilic polyurethane foams. Foams produced by currently known processes have tended to have relatively large cell sizes.

Absorbent foams for absorbent products have also been made from High Internal Phase Emulsions (hereafter referred to as “HIPE”). Open cell HIPE foams can provide the fluid capillary pressure necessary to remove most of the menstrual fluid from the body, or topsheet adjacent the body, thus minimizing wetness.

HIPE foams intended for absorption of blood and blood-based fluids can be formed into a single piece catamenial pad. Substantially planar HIPE foam-containing absorbent articles for absorption of blood and blood-based fluids are known.

As noted above, blood and blood-based liquids, such as menses are more highly viscous than water and especially urine. The higher viscosity of these liquids is further increased by the presence of electrolytes. Unfortunately, While HIPE foams, especially Thin-After-Drying (TAD) transport liquids extremely well, the rate of acquisition can be low.

It is desirable to provide absorbent components for absorbent articles, such as sanitary napkins, which are optimally soft and flexible and also optimally absorbent. It is still more desirable to produce such components from HIPE foams including the TAD (Thin After Drying) type, with a high rate of fluid acquisition. TAD foams have cells and holes small enough to provide a high capillary absorptive pressure but large enough to prevent or minimize blockage by the insoluble components of blood and blood-based liquids.

It is further desirable to improve the absorptive performance of HIPE foams by providing foams having apertures produced by a variety of means and embossments produced by selective expansion, both of which increase the permeability of the foam and therefore its absorption rate.

It is also desirable to improve the absorptive performance of HRPE foams by integrating the topsheet to the absorbent structure.

### SUMMARY OF THE INVENTION

The present invention relates to an absorbent article comprising an open-cell foam, which provides the absorbent structure thereof. The structure has an upper fluid-receiving surface, a lower surface opposite the upper surface, a perimeter edge, and a core region bounded by these surfaces. The structure has a means for enhancing transport of the fluids from the upper surface into the core region, the means being selected from: a) localized expanded regions of the foam, b) apertures in the foam; and c) integration to a topsheet and d) combinations of (a), (b), and (c). While preferred embodiments of the instant absorbent articles, are sanitary napkins and panty liners, it can be readily understood that absorbent articles for a broad range of uses, such as incontinent briefs, diapers, bandages etc., are additional examples.

Further, the present invention also relates to a process of treating thin-after-drying foam absorbent materials to provide selective expansion comprising the steps of: a) providing a foam absorbent structure, b) treating the foam structure with radiative or convective heat to reduce the moisture content in selected areas of the foam, c) where required providing a means of removing condensed water.

In another aspect, the present invention relates to a process for aperturing thin-after-drying foam absorbent materials comprising the steps of: a) providing a thin-after-drying foam absorbent structure, b) treating the foam structure with a means, which will impose a pattern of apertures on at least one surface thereof, the method being selected from compression cutting, sandblasting, laser cutting, airjet, waterjet, needling, drilling, punching, and ultrasonics.

### BRIEF DESCRIPTION OF THE DRAWINGS

FIG. 1 illustrates a cross-section of TAD foam which has been selectively expanded and apertured.

FIG. 2 shows a cross-section of TAD foam with an individual aperture through a selectively expanded portion prior to reexpansion.

FIG. 3 shows a cross-section of TAD foam with an individual aperture through a selectively expanded portion following reexpansion of the foam.

### DETAILED DESCRIPTION OF THE INVENTION

The “x” and “y” directions utilized in the “XY pattern” of the embossments and apertures of the instant articles are defined as follows. The absorbent article has a longitudinal centerline L which runs along the “x” axis. The term “longitudinal” as used herein refers to a line, axis or direction in the plane of the absorbent article that is generally aligned with (e.g., approximately parallel to) a vertical plane which bisects a standing wearer into left and right body halves when the absorbent article is worn. The “length” of the absorbent article is the linear measurement of the absorbent article in the x-direction.

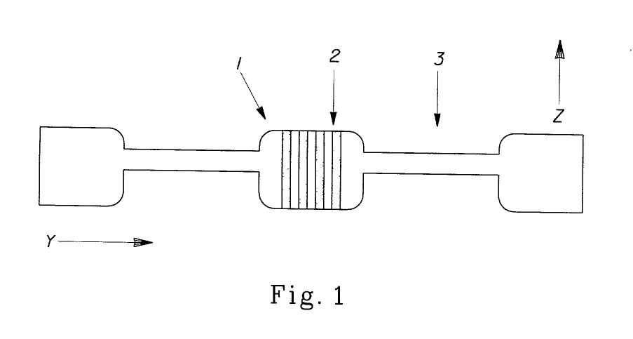

The transverse, lateral or “y direction”, as used herein, refers to a line, axis or direction that is generally perpendicular to the longitudinal direction. The lateral direction is shown in FIG. 1 as the “y” direction. The “width” of the absorbent article is the linear measurement of the absorbent article taken in the y-direction.

As used herein the “z” direction, shown in FIG. 1, is a direction parallel to the vertical plane described above. The “depth” or “height” of the absorbent article is the linear measurement of the absorbent article taken in the z-direction.

As used herein “upper” refers to an orientation in the z-direction toward the wearer's head.

As used herein “lower” or downwardly refers to an orientation in the z-direction toward the wearer's feet.

As used herein, “aperture” means an opening or open space.

As used herein “embossment” means a pattern element raised in relief from a surface.

As used herein, “sand blasting” means treating with a stream of sand or other particles projected by air or steam or other propulsion means.

As used herein “compression cutting” means cutting by pressing or squeezing a cutting tool into material to be cut.

As used herein “laser cutting” means cutting by means of light amplification by stimulated emission of atoms or molecules between energy levels for generating coherent electromagnetic radiation in the ultraviolet, visible or infrared regions of the spectrum.

Foam materials and foam absorbent structures treated according to the methods disclosed herein are particularly suitable for use as absorbent components in absorbent articles such as sanitary napkins, panty liners, interlabial devices, diapers, and adult incontinence pads to provide increased softness and conformability while providing high levels of absorbency.

In a preferred embodiment the absorbent article is comprised of a “thin after drying” (TAD) foam absorbent material. Such foam absorbent materials have cells and holes small enough to provide a high capillary absorptive pressure but large enough to prevent or minimize blockage by the insoluble components of blood and blood-based liquids such as menses. This structure provides foam materials capable of absorbing such liquids and then moving these absorbed liquids efficiently to other regions of the foam.

The starting TAD foam absorbent material has a capillary pressure, which is greater than its mechanical compression strength. The result is a material with a dry caliper 4 to 10 times lower than its wet caliper. The capillary pressure is created by the presence of water held in the structure and usually stabilized by a deliquescent material (preferably calcium chloride).

It has been found that the presence of apertures into or through the thickness of a homogeneous TAD foam absorbent increases its permeability and, therefore, its absorption rate. Such apertures can be created in a variety of ways, as disclosed below.

Additionally, embossments can be formed by reducing the moisture content (i.e., removing the water held in the structure) in selected areas of the foam. When the moisture content is reduced in selected areas the foam expands creating a semipermanent compression “XY pattern”. Such embossment is referred to as selective expansion.

In the area of the selectively expanded foam absorbent material, the cells in the expanded region are decompressed and have generally larger void volume than the cells of the unexpanded foam. These structural changes in the foam alter its fluid handling characteristics. Following selective expansion, the expanded area acquires fluid more rapidly than the unexpanded area, as would be expected from the larger void volume, but will preferentially release the fluid readily to the smaller cells of the unexpanded area. The “embossed” or “puffed” area of the foam is thicker than the untreated areas and can also be utilized around the perimeter edge of the foam pad to form a raised lip. This lip creates a “bowl” shape and is an aid to containment of fluids. This expanded perimeter edge is also useful in stopping fluid transport before it reaches the edge of the core thereby improving performance. The embossed or puffed areas can also be placed in such a manner that bunching of the absorbent article is minimized.

Preferred embodiments of the absorbent articles disclosed herein comprise a topsheet, a backsheet and an absorbent structure.

The Absorbent Structure

The foams used in the absorbent structure of the present invention are open-celled polymeric foams. For purposes of the present invention, a foam material is “open-celled” if at least about 80% of the cells in the foam structure that are at least 1 μm size are in liquid communication with at least one adjacent cell. The foams used in the foam absorbent structure of the present invention preferably have a number average cell size of from about 20 to about 250 μm. The cells in such substantially open-celled foam structures have intercellular openings or holes that provide passageways large enough to permit free and ready movement of blood and blood-based liquids, such as menses, from one cell to another within the foam structure, even though these liquids contain certain insoluble components. These substantially open-celled foam structures will generally have a reticulated character with the individual cells being defined by a plurality of mutually connected, three dimensionally branched struts. Cell size is a foam parameter that can impact a number of important mechanical and performance features of the absorbent foams used in the present invention. Cell size contributes to capillary suction specific surface area (CSSA), together with foam hydrophilicity, determines the capillarity of the foam. Therefore, cell size is a foam structure parameter that can directly affect the fluid wicking properties of absorbent foams, as well as the capillary pressure that is developed within the foam structure. A number of techniques are available for determining the cell size of foams. The most useful technique for determining cell size in foams involves a simple measurement based on the scanning electron photomicrograph of a foam sample. Superimposing a scale on a photomicrograph of the foam structure can be used to determine average cell size via visual inspection or an image analysis procedure. Foam cells, and especially cells that are formed by polymerizing a monomer-containing oil phase that surrounds relatively monomer-free water-phase droplets, will frequently be substantially spherical in shape. The size or “diameter” of such spherical cells is a commonly used parameter for characterizing foams in general. Since cells in a given sample of polymeric foam will not necessarily be of approximately the same size, an average cell size, i.e., number average cell diameter, will often be specified.

The cell size of HIPE foams for acquisition is preferably greater than that of the foam comprising for storage. Preferably, the cell size for acquisition foam (expressed in terms of number average cell diameter or mean cell diameter) ranges between about 100 and about 250 microns and the cell size for storage preferably ranges between about 20 to about 100 microns. The larger cell size provides the acquisition foam with the ability to acquire blood-based liquids at a higher rate by allowing red blood cells, debris, and other liquids to be taken up. The difference in cell size between an acquisition foam and a storage foam can establish a capillary gradient from the acquisition to the storage foams when both materials are a component of an absorbent structure. This will cause liquids to move from the acquisition portion into the storage portion. The movement of liquids out of the acquisition portion will drain the acquisition portion to make room in the acquisition portion for subsequent loading of liquids. In addition, the capillary gradient will also ensure that liquids which are transported to the storage portion will remain in the storage portion, and will not tend to go back up into the acquisition portion. The storage portion develops higher capillary pressure, but will generally accept menstrual liquids at a slower rate than the acquisition portion. TAD foams are especially preferred for use as storage foam.

Another feature useful in defining these preferred foams is hole size. The holes are the openings between adjacent cells that maintain liquid communication between these cells. The foams used in the present invention have hole sizes sufficiently large to allow passage of the insoluble components of blood, especially the red blood cells, to avoid blockage of these liquid passages. The preferred technique for determining hole size is image analysis based on scanning electron micrographs of the foams as discussed above. Depending on intended use, the foams used in the present invention various ranges for number average aperture size. For example, a foam for acquisition will suitably have cells ranging between about 20 μm and about 60 μm, preferably between about 30 μm and about 50 μm. Storage material has smaller cells with an average size between about 5 μm to about 40 μm, and preferably from about 10 to about 30 μm. As will be recognized, foams intended for use as an acquisition component generally have larger cells than foams intended for storage.

It may also be more desirable and preferable to alternatively express the difference in the foam properties of an acquisition portion and a storage portion in terms of “capillary specific surface area” (“CSSA”) since such a measurement may more accurately correlate with the liquid handling properties when two such portions are used in an absorbent structure. The capillary specific surface area is one of a number of characteristics important to absorbing and transporting blood and blood-based liquids. “Capillary specific surface area” is a measure of the test-liquid-accessible surface area of the polymeric network accessible to a test liquid. Capillary specific surface area is determined both by the dimensions of the cellular units in the foam and by the density of the polymer comprising the foam. It is, thus, a way of quantifying the total amount of solid surface provided by the foam network to the extent that such a surface participates in absorbency. The capillary specific surface area is determined by the method set forth in the TEST METHODS section of U.S. Pat. No. 5,387,207 issued to Dyer, et al. on Feb. 7, 1995 and is expressed in units of m²/cubic centimeter.

Generally, the CSSA of the foam at a constant volume increases as the cellular structure becomes smaller celled (or “finer”). Higher surface areas are highly desirable to develop the capillary pressure needed to attract liquids such as menses away from the body. However, the surface area of the foam can reach the point that the rate of liquid absorption becomes limiting, as well as increasing the likelihood that insoluble components within the liquid can no longer pass readily from one cell to another. Accordingly, the surface area of the foam needs to be selected within a particular range to balance these competing factors. Polymeric foams that are useful in the foam absorbent structure of the present invention are those that have a capillary specific surface area in the range of from about 0.0060 to about 0.10 m²/cc. Typically, the capillary specific surface area is in the range from about 0.010 to about 0.030 m²/cc, preferably from about 0.008 to about 0.04 m²/cc.

An acquisition portion of a multi portion structure preferably has a lower capillary specific surface area than a storage portion. For example, the acquisition portion may have a CSSA of from about 0.008 to about 0.020 m²/cc. The storage portion may have a capillary suction specific surface area, for example, of from about 0.020 to about 0.03 m²/cc. In this way, the storage portion will have a higher capillary pressure, allowing it to drain liquids from the acquisition portion, thus keeping the body of the wearer relatively free from contact with liquids.

The foams must be suitably resistant to deformation or compression by forces encountered when such absorbent foams are engaged in the absorption and retention of liquids. The resistance to compression deflection (or “RTCD”) exhibited by the polymeric foams used in the present invention can be quantified by determining the amount of strain (percentage of uncompressed height) produced in a sample of saturated foam held under a certain pressure for a specified period of time. The method for carrying out this particular type of test is described in the TEST METHODS section of U.S. Pat. No. 5,387,207, issued to Dyer, et al. Foams useful as absorbent members for catamenial products are those which exhibit a RTCD such that a confining pressure of 0.74 psi (5.1 kPa) at 31° C. after 15 minutes produces a strain of typically from about 5 to about 85% compression of the foam structure.

It may be desirable for at least a portion of the absorbent structure to compress to fit comfortably in the space between the wearer's labia and gluteal groove. It is estimated that the absorbent structure will not uncomfortably deform the wearer's labia if it has a RTCD that is between about 60% and about 80%. For multi portion absorbent structures the acquisition portion should have the same RTCD but a storage portion does not need to be as compressible if it is not in as close proximity to the wearer's body. In addition, providing a higher resistance to compression to a storage portion reduces any tendency for liquids to be “squeezed” out of the storage portion. The acquisition portion may, for example, have a RTCD of between about 60% to about 90%, and more preferably between about 75% to about 85%. The storage portion may, in such a case, have a RTCD of between about 5% to about 75%, and more preferably between about 35% to about 70%.

The foams used in the absorbent structure are preferably also sufficiently resilient so that they do not permanently collapse during use. This will ensure that the foams are able to continue to absorb bodily exudates throughout a wear cycle. The resilient characteristics of the foams can also help to ensure that the primary absorbent component will be capable of continuing to conform to and fill the space between the wearer's labia and gluteal groove after initial compression and after changes in the configuration of these parts of the wearer's body caused by body movements. Preferably, the foams used in the absorbent structure will return to at least about 70% of their uncompressed height, more preferably at least about 80%, and most preferably at least about 90% after the removal of the compressive forces.

Another important property of absorbent foams used in the present invention is their free absorbent capacity. For absorbent members useful in catamenial products, free absorbent capacity is the total amount of test liquid (i.e., synthetic urine) that a given foam sample will absorb at equilibrium into its cellular structure per unit mass of solid material in the sample. The foams that are especially useful as absorbent members in catamenial products will at least meet a minimum free absorbent capacity. The free absorbent capacity of the foams used in the present invention can be determined using the procedure described in the TEST METHODS section of U.S. Pat. No. 5,387,207 issued to Dyer, et al. To be especially useful as absorbent members for catamenial products, the foams used in the present invention should have a free absorbent capacity of from about 15 to about 125 g/g, preferably from about 20 to about 50 g/g, and most preferably about 25 g/g, of synthetic urine per gram of dry foam.

It should be understood that these foams can have different properties, features and/or characteristics at different times prior to contact between the foam and the blood or blood-based liquid to be absorbed. For example, during their manufacture, shipping, storage, etc., these foams can have density and/or cell size values outside the ranges set forth hereafter for these parameters, for example if they are stored in a compressed state by packaging. However, such foams are nevertheless still within the scope of this invention if they later undergo physical changes so that they have the requisite values specified hereafter for these properties, features and/or characteristics at least some point prior to and/or during contact with the blood or blood-based liquid to be absorbed.

While the absorbent structure forms an integral pad, in a preferred embodiment the absorbent structure of the article is multilayered, at least one layer of which is comprised of TAD foam. In a more preferred embodiment, the absorbent structure of the article is multilayered with at least one layer which is comprised of TAD foam and another which is a large celled foam. In this more preferred embodiment, the large celled foam layer would be positioned nearer the upper fluid-receiving surface.

Additionally, the foams disclosed herein can be hydrophilized by the addition of a combination of deliquescent salt and/or surfactant/emulsifier.

The absorbent structure can additionally comprise any material used in the art for such purpose. Non-limiting examples include natural materials, including comminuted wood pulp, which is generally referred to as airfelt, creped cellulose wadding, hydrogel-forming polymer gelling agents, modified cross-linked cellulose fibers, absorbent foams, absorbent sponges, synthetic staple fibers, polymeric fibers, peat moss or any equivalent material or combinations of materials.

The Topsheet

Topsheets optionally utilized by the instant invention are comprised of liquid pervious components, which permit liquids to readily penetrate the thickness. In more preferred embodiments the topsheet is hydrophobic. In order to function properly, the topsheet and absorbent structure must be in sustained fluid communication.

While nonwovens are preferred, a suitable topsheet can be manufactured from a wide range of materials such as woven and nonwoven materials; polymeric materials such as apertured formed thermoplastic films, apertured plastic films, and hydroformed plastic films; porous foams, reticulated foams; reticulated thermoplastic films; and thermoplastic scrims. Suitable woven and nonwoven materials can be comprised of synthetic fibers (e.g., polymeric fibers such as polyester, polypropylene, or polyethylene fibers). Such suitable topsheets may also be composite structures comprising both a formed thermoplastic film layer and a fibrous layer or two thermoplastic film layers that are subsequently formed.

The Backsheet

The optional backsheet prevents the exudates absorbed and contained in the absorbent structure from wetting articles, which contact the sanitary napkin. The backsheet may comprise a woven or nonwoven material, polymeric films such as thermoplastic films of polyethylene or polypropylene, or composite materials such as a film coated nonwoven material. The backsheet is preferably impervious to liquids, but may permit vapors to escape from the absorbent structure (i.e., breathable).

Preferred embodiments of the instant articles comprise apertured and/or embossed foam materials. The methods of aperturing and/or embossing are disclosed hereinafter.

Selective Expansion Processes

As previously noted, embossments can be formed in the TAD foams by temporarily reducing the moisture content in selected areas. When the moisture content is reduced the foam expands creating a semipermanent compression XY pattern. Such embossment, which is referred to as selective expansion, essentially dedensifies the foam structure in the selected areas.

The purpose of such selective expansion is to increase permeability of the absorbent material in the fluid target zone while retarding fluid wicking to the perimeter of the absorbent structure of the absorbent article. An additional advantage is the improvement in wearing comfort impression produced when some areas are perceived as soft and “cushioned” while the low caliper (thinness) of the majority of the product is maintained. Still further, when some areas of an absorbent structure are expanded, “bunching” of the product during wear is reduced.

Permeability

Fluid acquisition speed increases with increasing permeability. Expanding TAD absorbent foam materials in the fluid target zone (acquisition zone) increases the speed of fluid acquisition.

Fluid Wicking

Fluid wicks from an area of low capillary pressure to an area of high capillary pressure. Expanding thin-after-drying absorbent foam materials from their thin state to an expanded state reduces capillary pressure and therefore inhibits the wicking of fluid to the expanded area. When a narrow band of TAD HIPE foam around the perimeter of an absorbent structure is expanded fluids stop wicking to the perimeter. The band can traverse the entire perimeter or portions thereof and can be continuous or intermittent.

Expansion of the foam material must be sufficiently complete to create a large capillary pressure change between thin and expanded areas. Doubling the caliper from the thin state (about 50% of full expansion for typical foam absorbents) is acceptable. 85% expansion is more desirable while at least 95% is most desirable. When the expansion is significantly less than 100%, the location of the unexpanded cells can be important. For acquisition speed the unexpanded cells should be as far from the fluid receiving surface as possible (on the lower surface, adjacent a backsheet). For wicking control ideally the unexpanded cells would not be connected to each other so they don't provide a wicking continuum, which could wick fluid into the expanded area. If the unexpanded cells are continuous, they should be located so as to have the least visible impact. The preferred location would be in the z dimension center of the expanded zone, less preferred would be on the lower surface (towards the backsheet), least preferred would be on the upper surface (near the topsheet).

As previously stated, selective expansion (puffing) is accomplished by rapid vaporization of water in the expansion area. This is done most efficiently when the moisture content is optimized. If too little water is present, it becomes difficult to prevent expansion from occurring outside the area targeted for expansion. If too much moisture is present it may be impossible to expand or the energy and time required will be greatly increased. A moisture content of 10% by weight is optimum for expansion processes.

The water is held in the foam by deliquescent salt, preferably hydrated calcium chloride. The salt level should be selected to give a moisture content near optimum. This appears to be about 5%. The presence of salt may also aid in maximizing puffing energy absorption for some processes (i.e., radio frequency, microwave).

Overheating can result in foams which are hydrophobic rather than hydrophilic. This hydrophobicity is caused by loss of emulsifier and water. If the overheating is too severe, discoloration of the foam and presence of a bad odor can occur.

Heat Activation

Reduction of the moisture in selected areas of the foam is accomplished by selective heat activation of a portion of the foam. The heat can be radiative or convective. For example, the foam can be compressed between a heated plate and a cool plate in which the heated plate is fitted with a pattern of insulation corresponding to the unexpanded zone of the form. This causes the water in the non-insulated areas to boil and vaporize. Best results are achieved when the cool plate is fitted with an absorbent, such as paper toweling, to absorb any condensate which might be created.

Aperturing Processes

Formation of apertures in foam absorbent materials increases the rate of fluid absorption. During drying of absorbent foam materials two phenomena can negatively impact the fluid absorption rate. Firstly, salt can accumulate at the surface of the foam. Excessive salt can cause an increase in the viscosity of blood, which slows absorption rate. Secondly, if the moisture and/or emulsifier content at the surface of the foam drops below a critical value, the fluid contact angle will increase, resulting in a slowing of fluid absorption rate. When absorbent foams are dried at high temperatures, this effect is more pronounced (or can be absent) when the foams are air-dried.

The water present in foam absorbent materials has a high level of electrolyte. As the foam absorbent material is washed at the end of the production process, the water, which is high in electrolyte, preferably CaCl₂, washes to the surface. Therefore the foam absorbent material at the surface tends to block absorption of blood rather than to absorb it. By aperturing the foam absorbent material, the lower electrolyte content interior is exposed, so that as the blood moves through the apertures, it is absorbed.

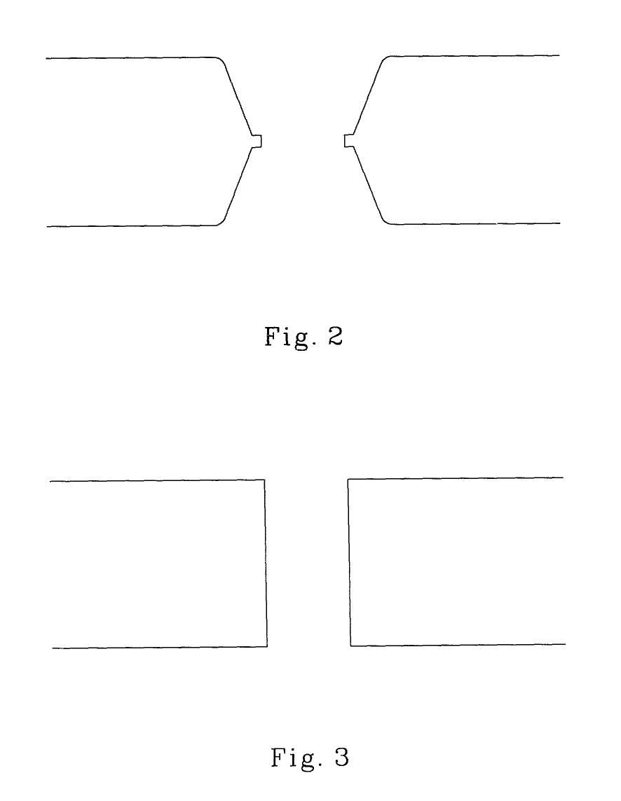

Apertures introduced into foam absorbent materials allow body fluids direct access to the faster absorbing interior portions of the foam, bypassing the more restrictive surface areas. It is, therefore, critical that the aperture be sufficiently open that fluid flow into the aperture and into the central zone of the foam absorbent structure is unimpeded. In order to ensure that the apertures function well, the foam absorbent material is either 1) removed from the aperture area, or 2) the foam struts in the aperture area are crushed such that the structural integrity is lost and the foam no longer fills the aperture void. The fact that these two processes aid aperture permeability, illustrate the importance of ensuring that the aperture perimeter is not further compressed. Processes which do not compress the foam, such as sand blasting, are acceptable. If an aperture process compresses the foam, for example, embossing or die cutting, the finished product criteria can be met if the foam is re-expanded, such as by “puffing” the apertured area. FIG. 2 shows an aperture produced by a compression process, while FIG. 3 shows an aperture following reexpansion of the surrounding foam.

Among the variables which can be controlled utilizing mechanical means are 1) aperture pattern, 2) size and density of apertures, 3) depth of penetration of the apertures into the foam absorbent structure, 4) shape of aperture, i.e., the cross section could be non-circular (oval, triangular, irregular, etc.).

While the apertures need not extend through the entire thickness of the absorbent structure, the flow resistant upper surface layer must be penetrated. This allows the fluid to directly reach the less resistant central zone. Apertures which are visible through the topsheet can enhance a users confidence in the absorptive capabilities of a product by providing a signal of increased absorbency. Such visibility can be achieved by means of making the apertures larger, ensuring that all material is removed from the aperture or creating a high level of color contrast between the absorbent material and the material immediately surrounding the aperture. When multiple layers of absorbent material are used visibility and performance are enhanced if the apertures in the layers of a multi-layered absorbent structure are aligned. The preferred size of each aperture is between 0.8 and 13 square millimeters. Further, The absorbent foam at the perimeter of an aperture preferably has a caliper no less than 80% of the caliper of absorbent foam surrounding the aperture.

Apertures change the overall compression strength of the foam, so aperture pattern can be selected to control softness and the “flexibility pattern” of the absorbent structure.

The central axis of the aperture need not be perpendicular to the foam surfaces. Additionally, while for some application, alignment of the apertures in various layers is preferred, the apertures can also be located differently in different layers of foam, i.e., a staggered pattern. Still further, the apertures can be tapered, being wider at one surface of the foam then where the aperture terminates.

The aperture process must not decrease the hydrophilicity of the foam. The most notable risk lies with a process that would heat the foam to a high temperature such that residual water and/or emulsifier are removed from the perimeter of the aperture. Laser cutting can produce such an effect.

Aperturing Methods

The process of aperturing the foam comprises the steps of:

a) providing a foam absorbent implement,

b) treating the foam implement with a means, which will impose a pattern of apertures, the method being selected from modified compression cutting techniques, sandblasting, laser cutting, airjet, waterjet, needling, drilling, punching, and ultrasonics,

c) removing the treatment means such that treatment of the foam implement is discontinued,

d) optionally, removing foam from the apertured area,

e) if necessary, reexpanding the foam immediately surrounding the aperture.

Compression cut apertures produced by traditional methods may produce foam materials, which do not absorb blood, because, the act of compressing seals the upper surface edge of the surface of the foam absorbent material to the lower surface edge of the foam absorbent material, so that the sides of the apertures had the same high electrolyte content as the surface of the foam absorbent material.

It has been found that sandblasting an aperture into the foam absorbent material allows blood-containing fluids access to the interior structure of the foam absorbent material. The interior of TAD foams has a lower electrolyte content and absorption of blood-containing fluids is improved. The sandblasting process does not seal the upper surface edge of the foam absorbent material to the lower surface edge of the foam absorbent material and therefore allows access to the interior, lower electrolyte, areas of the foam rather than the higher electrolyte upper and lower surfaces.

Additionally the apertures can be produced by other means for example, laser cutting or by ultrasonics.

Apertures can be provided uniformly throughout the absorbent structure or isolated to selected areas. An additional embodiment of the absorbent foam pad of this invention has multiple foam layers, which can be either simultaneously apertured or separately apertured with the same or different patterns, and then stacked.

Topsheet Integration

In preferred embodiments of the invention, glue, preferably sprayed hot melt adhesive, is applied between the topsheet and the upper surface of the foam absorbent structure. The topsheet/absorbent structure combination is pressed together with sufficient pressure to produce a strong glue bond and to transfer a small amount of surfactant/emulsifier, which has been added to hydrophilize the foam, from the foam to the topsheet. This attachment and compression results in a topsheet, which while hydrophobic, has hydrophilic portions on its lower surface. The compression is preferably done in a dot pattern wherein the dots have an area of from 0.8 to 28 square millimeters and the dots comprise 10% to 40% of the topsheet/absorbent structure contact area. Most preferably, the compression results in a dot pattern wherein the dots have an area of about 3 square millimeters and are uniformly distributed through the topsheet/absorbent structure contact area. The total dot area is optimally equal to about 20% of the topsheet/absorbent structure contact area. Compression in a dot pattern imparts a softer feel and appearance than does uniform compression.

The disclosures of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

While various embodiments and/or individual features of the present invention have been illustrated and described, it would be obvious to those skilled in the art that various other changes and modifications can be made without departing from the spirit and scope of the invention. As will be also be apparent to the skilled practitioner, all combinations of the embodiments and features taught in the foregoing disclosure are possible and can result in preferred executions of the invention. It is therefore intended to cover in the appended claims all such changes and modifications that are within the scope of this invention.

We claim:

1. An absorbent article comprising open-cell foam in the form of an absorbent structure, said structure having an upper fluid-receiving surface, a lower surface opposite said upper surface, a perimeter edge and a core region bounded by said surfaces, said structure having a means for enhancing transport of said fluids from said upper surface into said core region, said means being selected from the group consisting of:
a) localized expanded regions of said foam;
b) apertures in said foam said apertures having a perimeter;
c) integration of the upper surface with a topsheet; and
d) combinations of (a), (b), and (c).

2. The absorbent article according to claim 1 wherein said apertures pass through the entire core of said absorbent structure.

3. The absorbent article according to claim 1 wherein said apertures terminate within the core of said absorbent structure.

4. The absorbent article according to claim 1 wherein each of said apertures has an area of 0.8 to 13 square millimeters.

5. The absorbent article according to claim 1 wherein absorbent foam at the perimeter of said apertures has a caliper no less than 80% of the caliper of absorbent foam surrounding said aperture.

6. The absorbent article according to claim 1 wherein said expanded region forms a band around the perimeter edge of said absorbent structure.

7. The absorbent article according to claim 6 wherein the band is intermittent.

8. The absorbent article according to claim 1 wherein the open-cell foam is a High Internal Phase Emulsion (HIPE) foam.

9. The absorbent article according to claim 8 wherein the open cell foam is a Thin-After-Drying HIPE foam.

10. The absorbent article according to claim 1 wherein the absorbent structure is an integral pad.

11. The absorbent article according to claim 1 wherein the absorbent structure is a multilayered pad.

12. A process of embossing open cell foam absorbent materials by selective expansion comprising the steps of:
a) providing a TAD foam absorbent material,
b) treating the foam material by radiative or convective heat thereby reducing the moisture content in selected areas of the foam.

13. The process according to claim 12 further comprising a step wherein a means is provided for the removal of condensed water.

14. A process for aperturing open cell foam absorbent materials comprising the steps of:
a) providing a TAD foam absorbent structure,
b) treating the foam implement with a means, which will impose a pattern of apertures, the method being selected from compression cutting, sandblasting laser cutting, airjet, waterjet, needling, drilling, punching, and ultrasonics.

15. The process according to claim 14, further comprising the step of removing the treatment means such that treatment of the foam structure is discontinued.

16. The process according to claim 14 further comprising an additional step of reexpanding the apertured foam.

17. A process for integration of an absorbent structure of open cell foam, having an upper fluid-receiving surface, to a topsheet, having a lower surface, comprising the steps of:
a) providing the absorbent structure and the topsheet
b) applying glue between the topsheet and the upper surface of the absorbent structure
c) applying sufficient pressure to transfer an effective amount of surfactant/emulsifier present on the upper surface of the absorbent structure to the lower surface of the topsheet.

18. The process according to claim 17 wherein the pressure is applied in a dot pattern.
